(19) United States
(12) Patent Application Publication
(10) Pub. No.: US 2023/0075917 A1
(43) Pub. Date: Mar. 9, 2023

(54) Stimulating the Hairy Skin Through Ultrasonic Mid-Air Haptic Stimulation

(71) Applicant: Ultraleap Limited, Bristol (GB)

(72) Inventors: Dario Pittera, Bristol (GB); William Frier, Bristol (GB); Orestis Georgiou, Larnaca (CY); Sue Ann Seah, Bristol (GB); Benjamin John Oliver Long, Bristol (GB)

(21) Appl. No.: 17/822,224

(22) Filed: Aug. 25, 2022

Related U.S. Application Data
(60) Provisional application No. 63/238,215, filed on Aug. 29, 2021.

(51) Int. Cl. G10K 11/34; G06F 3/01

(52) U.S. Cl. CPC G10K 11/34; G06F 3/016

(57) ABSTRACT

Use of ultrasound beams to unlock the perception of mid-air tactile stimuli on hairy skin by using acoustic streaming is described. Such acoustic streaming is directional, more focused than fan and air-puff devices, doesn't require the use of cumbersome attachments, fast since it can be generated at a distance with the speed of sound, and can be used in a complementary fashion with vibrotactile mid-air stimulation deriving from the same hardware. Further proposed is a way to modulate the thermal perception of the mid-air tactile stimuli without the need for a thermal source or wearables. The effect of acoustic streaming and its thermal modulation may be combined for the accurate elicitation of affective touch sensations.

### PRIOR APPLICATION

This application claims the benefit of U.S. Provisional Patent Application No. 63/238,215, filed on Aug. 29, 2021, which is incorporated by reference in its entirety.

### FIELD OF THE DISCLOSURE

The present disclosure relates generally to stimulating hairy skin in mid-air haptic systems.

### BACKGROUND

Mid-air haptics is currently limited to vibration-based sensations. However, the major vastity of our skin is populated by tactile receptors tuned for non-vibrational stimuli. Furthermore, touch also encompasses dimensions such as affective touch and temperature perception. Affective touch has been defined as tactile processing with a hedonic or emotional component and C-tactile (CT) fibers are likely to convey this component. CT afferents are mostly found in the human hairy skin.

The questions discussed herein include:

1) Can modulated or focused ultrasound be used to induce an affective touch sensation on the hairy skin by exploiting acoustic streaming, a non-vibrational phenomenon?

2) Further, can the perceived temperature of a mid-air tactile stimulus induced through acoustic streaming be modulated?

3) Finally, can acoustic streaming and temperature to induce an affective stimulus be controlled?

Current solutions to these questions are described below.

### I. Stimulating the Hairy Skin Through Ultrasonic Mid-Air Touch

Eliciting touch through ultrasonic mid-air touch has been possible through the use of modulated waves on the glabrous (smooth) skin. Classically, these techniques have exploited amplitude modulation (AM) and spatio-temporal modulation (STM) (or lateral modulation (LM)). Because of the nature and the location of the mechanoreceptors in the skin sensitive to high-frequency stimuli, these techniques only enables the ultrasonic mid-air stimulation on the glabrous skin (i.e., hands, feet, lips, and genitalia). This means that the vast majority of our skin, the so-called hairy skin, remains a huge unexploited target area.

There are 3 references that could be located that used ultrasonic mid-air tactile stimulation on the hairy skin: Takahashi, R., Hasegawa, K., & Shinoda, H. (2020). Tactile Stimulation by Repetitive Lateral Movement of Midair Ultrasound Focus. IEEE Transactions on Haptics, 13(8), 334-342. (“Takahashi”); Gil, Hyunjae, et al. “Whiskers: Exploring the use of ultrasonic haptic cues on the face.” Proceedings of the 2018 CHI Conference on Human Factors in Computing Systems. 2018 (“Gil”); Kai Tsumoto, Tao Morisaki, Masahiro Fujiwara, Yasutoshi Makino and Hiroyuki Shinoda, (2021) Presentation of Tactile Pleasantness Using Airborne Ultrasound. Proceedings of World Haptics Congress 2021 (“Tsumoto”). These references focus respectively on the forearm and face. Nevertheless, to achieve a perceivable sensation on the hairy skin, these three previous works exploit specific stimulus modulation techniques (Takahashi—LM; Gil—AM/STM—not specified; Tsumoto—AM & stroking speed) and the acoustic radiation force exerted at the focus of the stimulation. Additionally, Takahashi used an array of 996 ultrasonic transducers (TXs). Gil, by attempting the stimulation on the face using acoustic radiation force as a main means of stimulation, required the use of eye and ears protectors. Overall, these solutions appear cumbersome and unpractical in ecological scenarios.

### II. Temperature Modulation Through Acoustic Streaming

There are two phenomena associated with an ultrasonic mid-air tactile stimulus: acoustic radiation pressure (ARP) and acoustic streaming (AS). Acoustic radiation pressure is a physical phenomenon resulting from the interaction of an acoustic wave with an obstacle placed along its path. Acoustic streaming is a nonlinear phenomenon, a result of momentum exchange between an acoustic field and the propagation medium (air), thus causing a steady airflow.

Generating acoustic streaming using ultrasound phased arrays has been previously demonstrated by Hasegawa, Keisuke, et al. “Electronically steerable ultrasound-driven long narrow air stream.” Applied Physics Letters 111.6 (2017): 064104 (“Hasegawa 2017”) and Hasegawa K, Yuki H, Shinoda H. Curved acceleration path of ultrasound-driven air flow. Journal of Applied Physics. 2019 Feb. 7; 125(5):054902 (“Hasegawa 2019”).

It has also been proposed for “transportation of gaseous substances”, e.g., for scent delivery purposes. Hasegawa K, Qiu L, Shinoda H. Midair ultrasound fragrance rendering. IEEE transactions on visualization and computer graphics. 2018 April; 24(4): 1477-85.

Acoustic streaming has also been employed to transport warm and cool air and provide thermal feedback to the user. The effect can be further exacerbated when the user wears a specific glove. Takaaki Kamigaki, Shun Suzuki, and Hiroyuki Shinoda, “Mid-air Thermal and Vibrotactile Display Using Focused Airborne Ultrasound,” International Conference on Human Haptic Sensing and Touch Enabled Computer Applications (Euro Haptics), Sep. 6-9, 2020, Leiden, Netherlands. However, this approach relies heavily on the presence of a heat source and the use of wearables.

### III. Affective Touch Through Ultrasonic Mid-Air Tactile Stimulation

Literature on physiology indicates that the hairy skin is vastly innervated by a special type of fibers, C-tactile (CT) which are supposed to encode the affective aspect of touch. Olausson, Halm, Johan Wessberg, and Francis McGlone, eds. Affective touch and the neurophysiology of CT afferents. Springer, 2016. Eliciting affective touch has been possible through the use of contact haptic devices (i.e., affective haptics), such as vibrating pillows, or wearables. Experiments on the hairy skin have generally involved the use of brushes moved at specific stroke-like speeds.

Tsalamlal, Mohamed Yacine, et al. “Affective communication through air jet stimulation: Evidence from event-related potentials.” International Journal of Human-Computer Interaction 34.12 (2018): 1157-1168 (and another 4 papers from the same authors) are studies that investigated a non-contact haptic elicitation of affective touch, namely using air jets. They state that their “study suggests that air jet tactile stimulation could be an efficient stimulation strategy for human-computer affective interactions” in their conclusion. Their solution has not been productized.

The main drawback of using air jets for any form of tactile stimulation is that it requires an air compressor, which is heavy and bulky. Another set of drawbacks are that air jets are diffusive, slow (speed of air) and loud. Finally, to create a moving air jet so that a stroking effect is emulated, then the air nozzle must be somehow motorized. The motorized equipment used will heavily affect the spatiotemporal modulation capabilities of the end-apparatus.

### SUMMARY

This disclosure proposes the use of ultrasound beams to unlock the perception of mid-air tactile stimuli on hairy skin by using acoustic streaming. The use of acoustic streaming is directional, more focused than fan and air-puff devices, doesn't require the use of cumbersome attachments, fast since it can be generated at a distance with the speed of sound, and can be used in a complementary fashion with vibrotactile mid-air stimulation deriving from the same hardware. Further proposed is a way to modulate the thermal perception of the mid-air tactile stimuli without the need for a thermal source or wearables. The effect of acoustic streaming and its thermal modulation may be combined for the accurate elicitation of affective touch sensations.

Proposed herein are methods for: a) producing mid-air tactile stimulation on the hairy skin; b) modulating the perceived temperature of a mid-air tactile stimulus; and c) inducing affective touch sensation, all of them by using high-pressure acoustic fields.

In one of the simplest examples, a focused ultrasound beam, e.g., a focal point (FP), is created near—but not on—the user's skin using a phased array. Downwind from the ultrasonic FP, an acoustic stream (i.e., a narrow air jet) begins to form due to the high acoustic pressure gradient. Further downwind from the focus the air jet diffuses (spreads out) and induces a convective cooling effect until eventually decaying away. At some optimal distance which depends on the input parameters, the acoustic stream is both strong enough and broad enough to induce an affective touch sensation (see FIG. 6). The jet needs to be strong enough to exceed the minimum activation threshold of the receptors in the hairy skin, i.e., mechanoreceptors and CT afferents. The jet also needs to be broad enough (covering a large skin area) so that several afferents are activated therefore registering in the brain. The acoustic streaming can “cool down” the skin through convection. Hence, by varying the FP to skin distance, the FP perceived temperature may also be modulated. Finally, the “acoustic streaming brush” is then gently moved at an optimal speed in the activation range of the CT afferents by moving the ultrasonic focus along the target area (e.g., the forearm) to further modulate the affective touch sensation, e.g., an arousing or pleasant feeling.

### BRIEF DESCRIPTION OF THE DRAWINGS

The accompanying figures, where like reference numerals refer to identical or functionally similar elements throughout the separate views, together with the detailed description below, are incorporated in and form part of the specification, serve to further illustrate embodiments of concepts that include the claimed invention and explain various principles and advantages of those embodiments.

FIGS. 1A, 1B, and 1C show schematics of experiments as to how acoustic streaming diffuses and decays.

FIG. 2 shows an example use case for inducing affective touch during a mid-air tactile interaction.

FIG. 3 shows an experimental arrangement for the subjective study of affective touch.

FIG. 4 shows a schematic for inducing streaming and modulating temperature.

FIG. 5 shows a flowchart for a method for inducing and controlling affective touch sensations using an ultrasound source.

FIG. 6 shows a graph of “vibrotactile only,” “vibrotactile and affective,” and “affective only” data.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

The hairy skin covers more than 90% of the human body, Zimmerman, A., Bai, L., & Ginty, D. D. (2014). While hairy skin is populated with almost the same receptors present in the glabrous skin, their density is lower, leading to a lower spatial acuity, hence, tactile discrimination capability. The hairy skin is also innervated by hair units and field units, rapidly adapting receptors with large fields and numerous high-sensitivity spots which are very sensitive to hair movements. Vallbo, A. B., Olausson, H., Wessberg, J., & Kakuda, N. (1995). Receptive field characteristics of tactile units with myelinated afferents in hairy skin of human subjects. The Journal of Physiology, 483(3), 783-795.

Among the tactile receptors on the hairy skin, CT-afferents are low-threshold mechanoreceptors whose function seems related to the encoding of pleasant tactile information, or affective touch. Their connection with the insular cortex together with their optimal activation for stroke-like tactile stimuli support this hypothesis.

### I. Acoustic Streaming

Ultrasonic phased arrays can manipulate the acoustic field for different purposes. Concerning mid-air haptic technology, the main approach consists of manipulating the acoustic field to maximize the acoustic pressure at the desired location to produce a small positive force that pushes against the user's skin (also referred to as acoustic radiation force). For a planar or focus field, the acoustic radiation pressure P (in Pascals) on the skin surface can to a good approximation be related to the acoustic RMS pressure p (also in Pascals) through:

$$P = α \frac{{p}^{2}}{ρ {c}^{2}}$$

where c=343 m/s is the speed of sound in air, p=1.225 kg/m³ is the density of air, α=1+R² where R is the reflection coefficient of the interface. To induce a vibrotactile sensation, this pressure is usually modulated in time or space within a range of fundamental frequencies that stimulate the appropriate receptor fields such as the Pacinian mechanoreceptors. The modulations of the acoustic field are achieved through the appropriate control of the activation coefficients of the ultrasonic phased array transducers.

There are many ways of manipulating the acoustic field, including phased arrays, metamaterials, parabolic transducer arrangements, etc. Regardless of the method of generating localized high acoustic pressure points, the result of interest for affective touch is the acoustic streaming field, a by-product of high acoustic pressures. As described in Bruus, Henrik. “Acoustofluidics 2: Perturbation theory and ultrasound resonance modes.” Lab on a Chip 12.1 (2012): 20-28, the second-order velocity u₂, which is also known as the acoustic streaming field, may be defined with fluid density at rest ρ₀ and the acoustic perturbations in density and velocity due to the acoustic wave defined as ρ₁ and u₁ respectively as:

ρ₀∇· [symbol] u₂ [symbol] =−∇· [symbol] ρ₁u₁ [symbol] ,

where the angle brackets denote quantities averaged across acoustic cycles. By integrating over the volume under consideration and invoking the divergence theorem to exchange the volume integrals for surface integrals, this may be written as:

ρ₀ [symbol] ·\hat{n}dS=− [symbol] ·\hat{n}dS.

So, for the control volume Ω, this means that any change in (or absorption of) the averaged momentum flux carried by the acoustic wave across the boundary will result in contributions to the averaged acoustic streaming field across the boundary. Since there must exist a mechanism by which the averaged momentum flux carried by the wave is depleted as the acoustic wave travels for the acoustic streaming effect to be present, it follows that acoustic streaming is often defined as a steady flow driven by a soundwave in a viscous fluid. This is because viscosity is in many cases the dominant means by which momentum flux is removed from the wave. The greater the sound pressure, the faster the fluid flow, because as ρ₁ is the acoustic perturbation of density, it may be related to the acoustic pressure as p₁=c₀²ρ₁. In the far-field of the array, the plane wave approximation (from Landau, L. D. and Lifshitz, E. M. “Fluid mechanics” Pergamon Press (1963)) may be used to write:

$${u}_{1} = \frac{{c}_{0} {ρ}_{1}}{{ρ}_{0}} = \frac{{p}_{1}}{{c}_{0} {ρ}_{0}} ,$$

showing that in this case, the streaming effect scales with the square of the acoustic pressure.

Nonlinearities of the propagation medium (air or sometimes water) have essentially two main effects on streaming generation. First, they restrict the air jet to linear flow (non-turbulent). Second, they reduce the streaming velocity in the focal and pre-focal region whereas accelerate the flow in the post-focal region. In fact, the maximum point of AS velocity is located somewhat outside the FP. Kamakura, Tomoo, et al. “Acoustic streaming induced in focused Gaussian beams.” The Journal of the Acoustical Society of America 97.5 (1995): 2740-2746.

The bulk movement of air particles at velocities (u₂) can be related to a drag force F applied to the user's skin through:

$$F = \frac{1}{2} ρ \int {\int }_{A} {〈 {u}_{2} 〉}^{2} dxdy ,$$

where A is the area stimulated by the air stream. The spatial distribution of [symbol] u₂ [symbol] can be controlled by the sharpness of the FP beam. Moreover, the AS effect is not instantaneous but rather exhibits a slow build-up with a time constant in the order of milliseconds. This temporal time delay can be leveraged and used to design, modulate, and multiplex the tactile effects of AS. For example, the high-intensity FP can be amplitude modulated on and off (e.g., pulsing) to generate pulses of AS at a similar envelope frequency as the FP. This may even have the effect of creating a narrower air jet.

The phenomenon of acoustic streaming was first found by resonance in a tube by Faraday in 1831. A net flow associated with a vibrating plate was observed by Lord Rayleigh in 1884, noting that currents of air ascended at displacement antinodes and descended at the nodes of the plate. The flow in the inner boundary layer of the resonant tube was explained by Schlichting in 1932. Finally, Eckart in 1948 reported another type of streaming driven by the attenuation (e.g., momentum exchange) of sound waves. These three types of Rayleigh, Schlichting and Eckart streaming are known as the representative types of acoustic streaming and are well explained in the physics literature.

Acoustic streaming induced by ultrasound beams has been extensively studied. There are several advantages in using ultrasound to induce acoustic streaming effects. Primarily these include high directivity, inaudibility, and high sound pressure level.

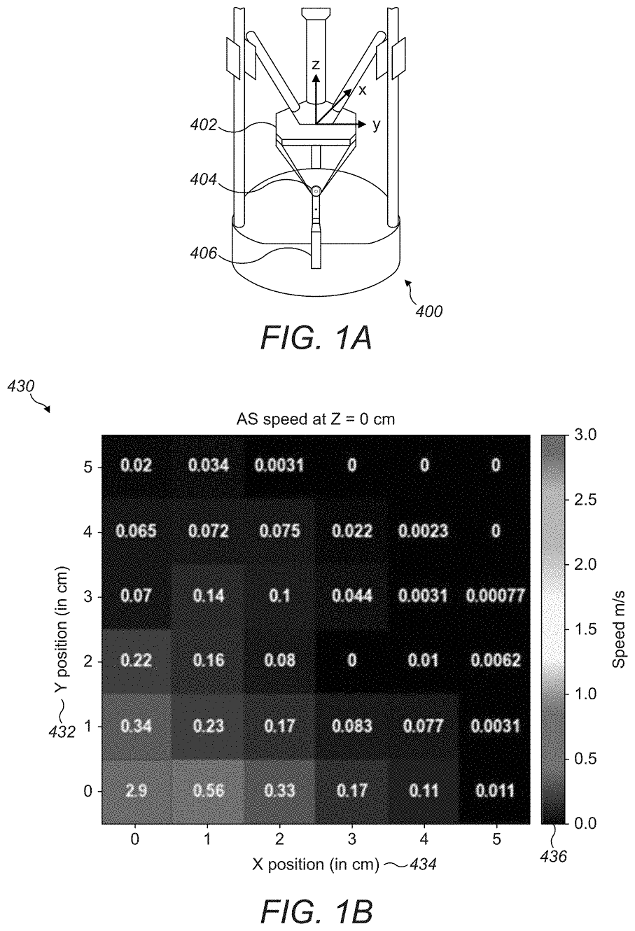

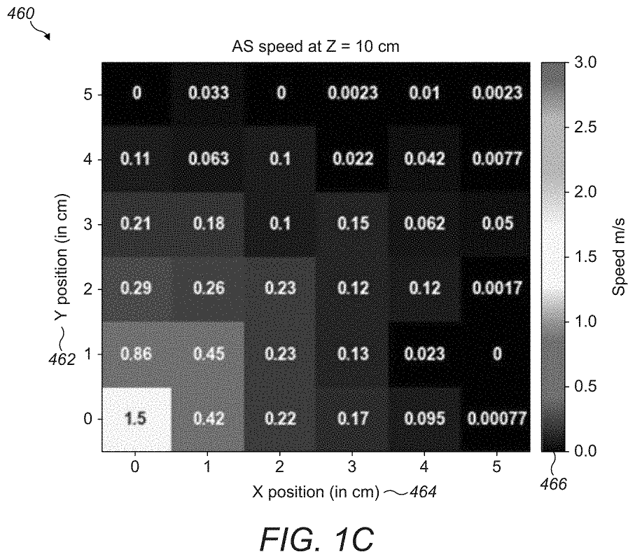

Acoustics streaming can be accurately modelled using computer simulation software. It is therefore known that the streaming flow velocity increases with high sound pressure gradients, while it has also been shown experimentally that acoustic nonlinearity (e.g., due to high viscosity) enhances streaming velocity. It has been reported that the massively arrayed pressure transducers used for haptic devices can produce 2-3 m/s acoustic streaming in the air (Hasegawa 2017 and FIGS. 1A, 1B, and 1C) and that the acoustic field can even be shaped as to produce a curved acceleration path in the air (Hasegawa 2019).

Ultrasonic phased arrays, therefore, can manipulate and maximize acoustic pressure gradients in the field, and therefore maximize acoustic streaming both in magnitude and spread. FIGS. 1A, 1B, and 1C shows the speed of the flow produced with acoustic streaming in the proximity of a zone of high acoustic pressure (FP) and a zone of low acoustic pressure (i.e., ambient pressure).

Specifically, FIGS. 1A, 1B, and 1C show experiments illustrating how acoustic streaming diffuses and decays at z=10 cm downwind from the focus at z=0. FIG. 1A shows a setup 400 used to measure the acoustic streaming speed. A hot wire anemometer 406 is placed downwind the focal point (FP) 404 created by the ultrasound device. The FP is located 20 cm from the array surface 402. The array 402 is then moved along the x, y, z axes to measure variations in acoustic streaming speed (in m/s).

FIG. 1B shows a graph 430 of acoustic streaming measurements for a 6×6 cm surface at z=0 (tip of the anemometer at the ultrasonic focus). The x-axis 434 is the X position in cm. The y-axis 432 is the Y position in cm. The scale 436 is speed in m/s.

FIG. 1C shows a graph 460 of acoustic streaming measurements for a 6×6 cm surface at z=10 cm (anemometer tip is 10 cm downwind from the ultrasonic focus). The x-axis 464 is the X position in cm. The y-axis 462 is the Y position in cm. The scale 466 is speed in m/s.

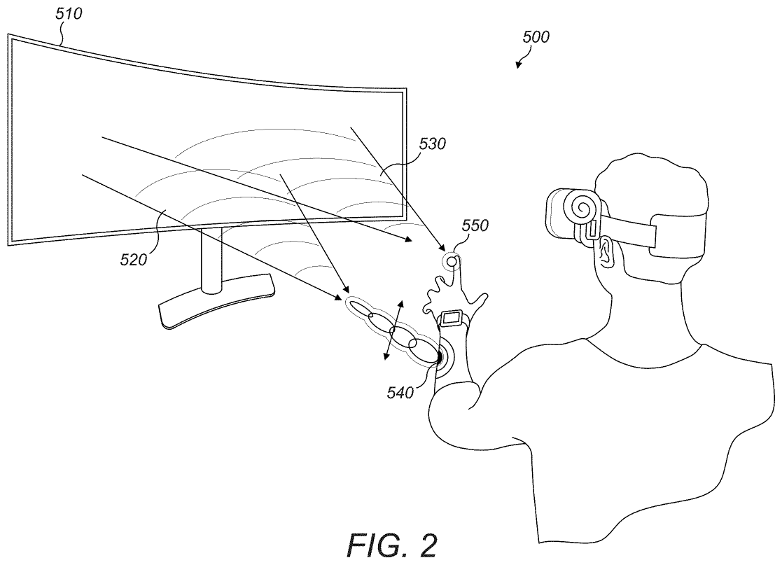

FIG. 2 shows that it is possible to use one unique ultrasonic phased-array (256 TXs against 996 TXs in Takahashi), to convey both, functional touch (due to direct vibrotactile stimulation) and affective touch (due to in-direct acoustics streaming stimulation) to the user on the hairy skin. Functional touch is conveyed using acoustic radiation pressure (ARP) stimuli and affective touch is conveyed using acoustic streaming (AS).

Specifically, FIG. 2 shows an example use case 500 for inducing affective touch 520, 540 during a mid-air tactile interaction from an ultrasonic phased array 510 in virtual reality. Traditional vibrotactile mid-air touch 530, 550 can be applied on the glabrous (non-hairy) parts of the skin to convey functional tactile information (e.g., notifications, action feedback, texture sensations, etc.). Meanwhile, hairy parts of the skin may be simulated to elicit affective tactile sensations on the same person or different people creating, for instance, social interactions for multiple users.

Further, because airflow is used to stimulate the hairy skin, the heat transfer from the skin to the air may be increased through convection streams, which leads the stimuli to be perceived as colder. This is a more efficient way compared to previous methods used to produce cooling effects (Nakajima M, Hasegawa K, Makino Y, Shinoda H. Remotely displaying cooling sensation via ultrasound-driven air flow. In 2018 IEEE Haptics Symposium (HAPTICS) 2018 Mar. 25 (pp. 340-343). IEEE.), which do not require the use of further attachments. By varying the hairy skin-FP distance, tactile stimuli which are felt at different temperatures may be created. In this way, the perceived temperature of a mid-air tactile stimulus may be modulated.

Moreover, it is possible to optimize the perceived affective touch sensation by considering the sensitivity of the CT-afferents. Mechanoreceptor sensitivity is a well-known property already used in applications for functional touch. For instance, Pacinian corpuscles, one of the mechanoreceptors involved in functional touch, have a frequency sensitivity. This means that the threshold needed to be exceeded before “firing” a signal to the brain is lower (close to peak sensitivity) or greater (far from peak sensitivity) depending on the stimulus frequency. Likewise, CT-afferents have a sensitivity to stroke-like stimuli speeds. Hence, a method to tune the stroke speed of “acoustic streaming brush” to the CT-afferent sensitivity is proposed herein. Literature and preliminary studies suggest a peak sensitivity between 1 and 10 cm/s, Loken, L. S., Wessberg, J., Morrison, I., McGlone, F., & Olausson, H. (2009). Coding of pleasant touch by unmyelinated afferents in humans. Nature Neuroscience, 12(5), 547-548, a speed within the capabilities of current ultrasonic phased arrays.

Meanwhile, research on human hairy skin is not comprehensive. But there have been several indications of the presence of Pacinian corpuscles in the hairy skin. Bolanowski, S. J., Gescheider, G. A., & Verrillo, R. T. (1994). Hairy skin: Psychophysical channels and their physiological substrates. Somatosensory & Motor Research, 11(3), 279-290; Lechner, S. G., & Lewin, G. R. (2013). Hairy sensation. Physiology, Vol. 28, pp. 142-150. Hence, the existence of spatial summation phenomena typical of these receptors may not be excluded. Therefore, the method for eliciting affective touch can further tailor the acoustic streaming stimuli to consider the “acoustic haptic brush” size. In this way, the spatial summation effect to increase the perceived strength of the stimulation on different parts of the hairy skin and modulate the stimulus' strength depending on the application needed and context may be exploited. Consequently, by being able to stimulate the hairy skin, the target area for stimulate may be broadened (e.g., forearms, legs). This allows tactile sensation even in situations where the hands are busy, as in an automotive context, during surgery, manual repairments, etc.

### II. Example Use Case

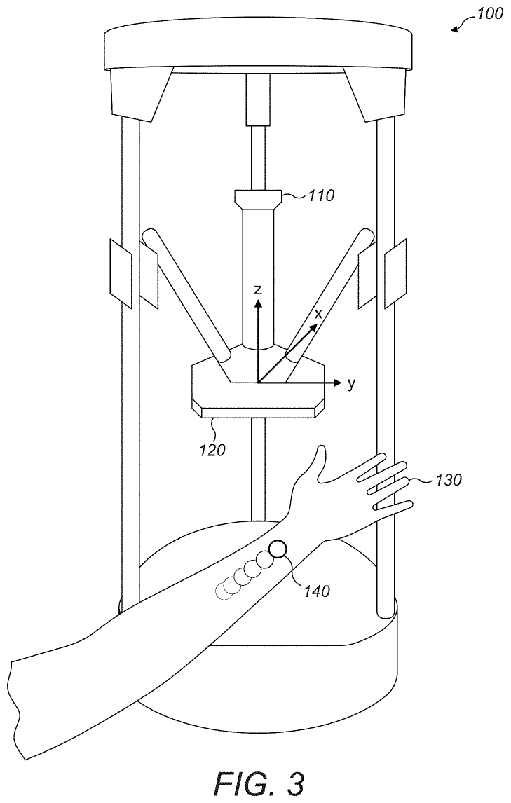

An example use case is a driver “Mary” coming back from her summer vacation. As Mary was not the only one taking advantage of the good weather, she ended up stuck in traffic under the scorching sun of August. Her car is equipped with proximity sensors to track the driver's target area and acquire its position in the environment. After detecting the pattern of driving by using the speed, the number of breaks, and meters travelled, Mary's smart car noticed a situation of traffic jam and it activates an array of ultrasonic transducers to deliver an ultrasonic beam on her forearm (as seen in FIG. 3).

Specifically, FIG. 3 shows an experimental arrangement 100 for the subjective study of affective touch. A hairy skin part of the body 130 is placed downwind to an ultrasound array 120. An XYZ robot 110 moves the array along the x, y, z axes. The ultrasonic focus 140 will be at distance from the robot, being perceivable from the observer. By varying the distance between hairy skin part of the body 130 and the ultrasonic focus 140, the perceived temperature of the stimulus may be controlled. Finally, the ultrasonic focus 140 can be moved at an optimal speed to convey affective touch-like mid-air tactile stimuli.

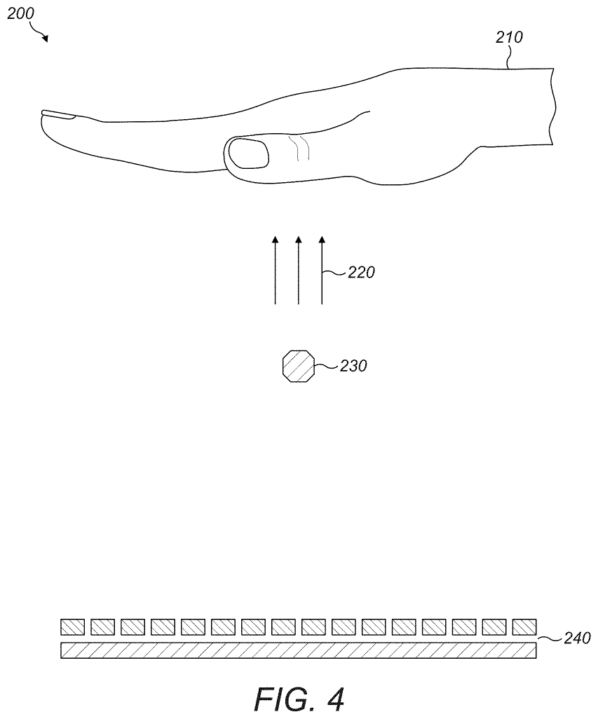

The software of the smart car has already some predetermined stimulation tailored for the ultrasonic device that has been shown to elicit different affective meanings. The library was built by modulating the FP speed in the range of CT-afferent fibers and by optimally controlling the distance between the FP and the hairy skin to create an acoustic streaming (FIG. 4) through the device SDK.

Specifically, FIG. 4 shows a schematic 200 for inducing streaming and modulating temperature. An array of ultrasonic transducers 240 render a focal point 230. Acoustic streaming 220 will be created above the focal point 230 that will be perceived on the target 210. By varying the distance between the target 210 and the focal point 230, the perceived temperature on the skin (glabrous and hairy) may be controlled. Additionally, by creating a second focal point on the target, both vibrotactile stimulation and thermal stimulation may be delivered at the same time.

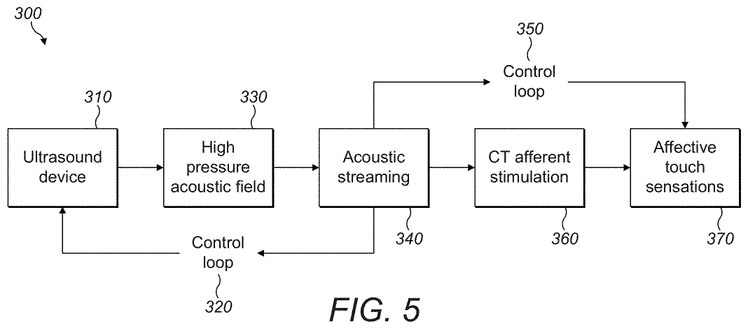

Now Mary receives a soothing tactile sensation to help her bear with the traffic jam. As described in FIG. 5, by modulating the high-pressure field through the device SDK the acoustic streaming produced may be modulated to finally alter the desired induced affective effect. In this way, later in her trip, when Mary will be tired, her smart car will produce a different mid-air haptic effect to elevate her attention level.

Specifically, FIG. 5 shows a flowchart 300 for a method for inducing and controlling affective touch sensations using an ultrasound source. An ultrasonic source 310 produces a high-pressure field 330 that generates acoustic streaming 340. By changing the acoustic pressure field, we can control the acoustic streaming 320. The generated acoustic streaming 340 can stimulate CT afferents 360 which can induce an affective touch sensation 370. By changing the acoustic streaming properties, we can control the affective touch sensations 350. The control loop blocks 320, 350 can be replaced by pre-calibrated recordings of the intended acoustic fields, output from a solver, and could also accept calibration input from external sensors and systems.

### III. Affective Touch Via Temperature Control Plus Acoustic Streaming

Finally, temperature control may be combined with the above-described application of acoustic streaming to elicit affective touch. It is known that the CT-afferents are more sensitive to skin temperature stimuli (i.e., around 30 degrees Celsius). Ackerley, R., Backlund Wasling, H., Liljencrantz, J., Olausson, H., Johnson, R. D., & Wessberg, J. (2014). Human C-tactile afferents are tuned to the temperature of a skin-stroking caress. Journal of Neuroscience, 34(8), 2879-2883. To mitigate this phenomenon, methods are proposed to modulate the acoustic streaming over time. This controls the convection phenomenon at the skin surface and maintains an optimal skin temperature while maintaining a high peak-velocity of the airflow.

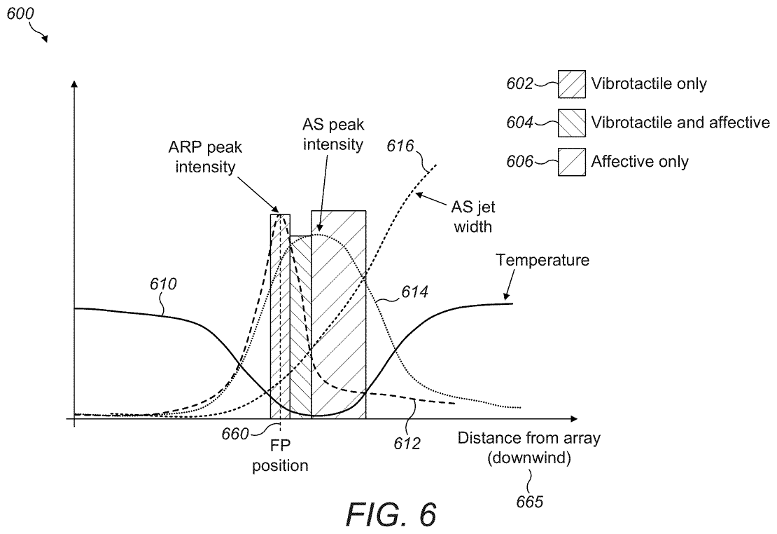

Varying the FP to target distance, enables the rendering of mid-air haptics at different perceived temperatures (see FIG. 4) and thus, creates control over the final affective sensation (see FIG. 5). In the previous example, the distance between the FP and Mary's forearm could be increased to deliver a fresh sensation while still modulating the FP speed in the range of the CT-afferents to stimulate affective touch. This interaction space can be summed up by FIG. 6, which shows how both vibrotactile and affective stimulations may be obtained with the same hardware device

Specifically, FIG. 6 shows a schematic 600 of “vibrotactile only” 602, “vibrotactile and affective” 604, and “affective only” 606 data. The x-axis 665 is distance from the phased array as in the setup shown in FIG. 4. The acoustic radiation pressure (ARP) 612 responsible for vibrotactile stimulation increases and then drops rapidly in the vicinity of the focal point (FP) position 660 as a function of the distance from the phased array. A similar trend is exhibited by the acoustic streaming (AS) 614, however, the bell-shaped curve is much wider than that of the ARP intensity 612. The drop in AS intensity 614 is proportional to the AS jet width 616 which increases in a diffusive manner. The temperature 610 drops in the center of the graph. The schematic, therefore, helps identify regions in space where vibrotactile, affective, and composite stimulation are optimal.

By emitting an FP, ARP is created and in consequence also AS. When the target area is at the focus of the ultrasonic beam it is possible to stimulate mainly the mechanoreceptors in the skin sensitive to mid-high frequencies vibrations. Moving farther from the FP, AS becomes the dominant stimulation factor being able to stimulate the CT-afferent in the hairy parts of the skin, passing from delivering vibrotactile and affective sensation to exclusively the latter. Together with the increase of AS, the temperature will be perceived as colder. This enables stimulation of the hairy skin at the optimal speed for affective touch and allows exploitation of the different perceived temperatures to strengthen the affective effect (as in the case of Mary being refreshed and calmed by her smart car).

Being able to successfully elicit affective touch, and in turn, social touch opens up a series of scenarios to increase our quality of life. It is known that affective touch plays a key role in the adult-baby dyad for the healthy development of the baby. And the role of affective touch and social touch remains crucial also during adulthood. Pleasant touch promotes affiliative behavior, familial affection, the creation of social bonds, emotion modulation, and positive behavior. Further, it can act as a stress-relief mechanism and it reduces the feeling of social exclusion. Morrison, I., Loken, L. S., & Olausson, H. (2010). The skin as a social organ. Experimental Brain Research, 204(3), 305-314; Von Mohr, M., Kirsch, L. P., & Fotopoulou, A. (2017). The soothing function of touch: Affective touch reduces feelings of social exclusion. Scientific Reports, 7(1). Previous research demonstrated how touch can increase the autonomic response of a romantic couple allowing the emergence of a somatovisceral synchronization that is supposedly at the base of emotional contagion and empathy behaviors. Chatel-Goldman, J., Congedo, M., Jutten, C., & Schwartz, J. L. (2014). Touch increases autonomic coupling between romantic partners. Frontiers in Behavioral Neuroscience, 8 (MAR).

Taken together and in the broader context of the Covid-19 pandemic that imposed touch deprivation, being able to convey affective and social touch at distance becomes of primary importance. This will be true even in an after-pandemic time. In those situations in which the beloved ones are physically distant and feel alone, modern technology allows for consistent visual stimuli (i.e., video calls, VR meetings, pictures exchange, etc.) But the lack of a tactile sensation emerges as a fundamental limitation. Allowing the rendering and delivering of affective and social touch would shorten the distance between people, ultimately, making the interaction “more real”. The elderly in the hospices, the beloved ones in the hospital, couples and family at distance, will be able to see and touch each other sustaining the positive effects of social touch we discussed above. Perhaps it will be possible to provide touch to premature babies who are enclosed in incubators at the early stage of their life, promoting a natural and healthy development. The communication of emotions and social touch between people is fundamental to human life and as long as there will be a distance between people, there will always be demand for reducing it.

Finally, it is worth noting that it is possible that affective touch is not coded only by the CT fibers, and that pleasantness is constructed in the central nervous system. Sailer, U., Hausmann, M., & Croy, I. (2020). Pleasantness only? How sensory and affective attributes describe touch targeting C-tactile fibers. Experimental Psychology, 67, in press. Hence, affective touch might be produced even in non-CT or sub-optimal stimulation and can be also highly reliant on contextual situations. This means, that by being able to modulate the perceived temperature of a mid-air tactile stimulus (even outside the optimal range for CT fibers), the affective touch system could still be successfully stimulated.

For instance, consider a scenario in which the user is located in a hot space. It could be a gym, the corridor of a tube station, a train carriage, or an airport hub with direct sun exposure and insufficient A/C. A mid-air stimulus delivered at distance will be felt as pleasantly cool, like a refreshing breeze through an invisible window, providing a pleasant relief onto the subject's skin and lowering the frustration of an early morning trip or a long waiting time. The cooling stimulus can be automatically started over a specific temperature threshold, or actioned and controlled by the user. It is highly directional, it can change shape, and it can be moved on the preferred spot.

Stimulating the hairy skin, while modulating the perceived temperature of the stimuli, and activating the CT-afferents have a threefold consequence. First, it unlocks mid-air haptics perception to other parts of the body, moving beyond the current limit of the palms (i.e., glabrous skin). Second, it unlocks an affective channel that enables a new range of applications and use-cases for emotional and social communication. Finally, by relying on more general means of activating the affective touch system that extends further from the CT fibers activity, temperature modulation may be used to enrich mid-air tactile stimuli with affective meaning.

### IV. Possible Points of Novelty

Advantages Over the Prior Art Include:

1. Temporal and Spatial control of acoustic streaming via the use of ultrasonic phased arrays to accurately stimulate the hairy skin in a controlled and contactless manner; and

2. No thermal source or wearables are required to modulate the perceived temperature of the mid-air tactile stimulation.

Possible Points of Novelty Include:

1. The use of ultrasound to stimulate the hairy skin and to create affective touch sensations that are not based on vibrotactile stimuli but instead leverage acoustic streaming; and

2. Leverage acoustic streaming to use ultrasound phased array as a thermal display, to allow tactile stimuli to be perceived at different temperatures.

3. A method comprising the use of a plethora of acoustic transducers to induce affective touch sensations by creating acoustic streaming air jets that are targeted towards the human skin; wherein the acoustic transducers focus acoustic waves so to form pressure gradients in the vicinity of the targeted skin.

4. Paragraph 3, further comprising of spatiotemporal modulation of the acoustic field gradients to control the acoustic streaming air jet's location, speed, perceived temperature, and/or shape relative to the targeted skin.

5. Paragraph 3, further comprising of temporal and spatial multiplexing with a plethora of modulated focused or unfocused control points for the delivery of composite affective and vibrotactile contactless sensations in mid-air.

### V. Conclusion

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. The term “coupled” as used herein is defined as connected, although not necessarily directly and not necessarily mechanically. A device or structure that is “configured” in a certain way is configured in at least that way but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1. A method comprising:
a plurality of acoustic transducers inducing affective touch sensations on targeted skin of a user by creating acoustic streaming air jets targeted towards the targeted skin;
wherein the plurality of acoustic transducers focus acoustic waves to form pressure gradients in vicinity of the targeted skin.

2. The method of claim 1, wherein the air jets do not directly touch the targeted skin.

3. The method of claim 2, further comprising:
spatiotemporally modulating acoustic field gradients to control the acoustic streaming air jet location relative to the targeted skin.

4. The method of claim 2, further comprising:
spatiotemporally modulating acoustic field gradients to control the acoustic streaming air jet speed relative to the targeted skin.

5. The method of claim 2, further comprising:
spatiotemporally modulating acoustic field gradients to control the acoustic streaming air jet perceived temperature relative to the targeted skin.

6. The method of claim 2, further comprising:
spatiotemporally modulating acoustic field gradients to control the acoustic streaming air jet shape relative to the targeted skin.

7. The method of claim 2, further comprising:
temporal and spatial multiplexing with a plurality of modulated control points for delivery of composite affective and vibrotactile contactless sensations in mid-air.

8. The method of claim 7, wherein at least one of the plurality of modulated control points is focused.

9. The method of claim 7, wherein at least one of the plurality of modulated control points is unfocused.

10. A system comprising:
an ultrasonic phased array that conveys functional touch to hairy skin of a user due to direct vibrotactile stimulation and affective touch to the hairy skin of the user due to direct acoustics streaming stimulation.

11. The system as in claim 10, wherein the vibrotactile sensation comprises pressure modulated in time within a range of fundamental frequencies that stimulate receptor fields.

12. The system as in claim 11, wherein the receptor fields are Pacinian mechanoreceptors.

13. The system as in claim 10, wherein the vibrotactile sensation comprises pressure modulated in space within a range of fundamental frequencies that stimulate receptor fields.

14. The system as in claim 13, wherein the receptor fields are Pacinian mechanoreceptors.

15. The system as in claim 10, further comprising:
a focal point that produces acoustic radiation pressure and acoustic streaming;
wherein the direct vibrotactile stimulation results from the acoustic radiation pressure.

16. The system as in claim 15, wherein when the hairy skin is moved away from the focal point, the affective touch will be stronger than the functional touch.

17. The system as in claim 16, wherein when the hairy skin is moved away from the focal point, temperature on the hairy skin will be perceived as colder.

18. A method comprising:
focusing an ultrasound beam as a focal point near skin of a user using a phased array, wherein the focal point is not on the skin of the user;
forming an acoustic stream as a narrow air jet downwind from the focal point due a high acoustic pressure gradient; and
diffusing the air jet downwind from the focal point, thereby inducing a convective cooling effect on the skin of the user.

19. The method as in claim 18, wherein the skin of the user is hairy skin, and wherein the air jet is strong enough to exceed minimum activation threshold of receptors in the hairy skin.

20. The method as in claim 19, further comprising:
modulating perceived temperature on the skin of the user by varying the distance between the focal point and the skin of the user.
